(19) United States
(12) Patent Application Publication
(10) Pub. No.: US 2021/0346173 A1
(43) Pub. Date: Nov. 11, 2021

(54) METHODS AND APPARATUSES FOR GRAPHIC PROCESSING IN A VISUAL DISPLAY SYSTEM FOR THE PLANNING AND EXECUTION OF FUSION OF THE CERVICAL SPINE

(71) Applicant: WENZEL SPINE, INC., Austin, TX (US)

(72) Inventors: Adam DEITZ, Austin, TX (US); Steve Won-Tze CHANG, Phoenix, AZ (US)

(73) Assignee: WENZEL SPINE, INC., Austin, TX (US)

(21) Appl. No.: 17/246,823

(22) Filed: May 3, 2021

Related U.S. Application Data
(60) Provisional application No. 63/022,639, filed on May 11, 2020.

(51) Int. Cl. A61F 2/46; G16H 30/20; G16H 30/40; G16H 50/30; G16H 20/40; G16H 40/20; G06T 7/20; G06T 7/00; G06T 7/60; G06T 11/20

(52) U.S. Cl. CPC A61F 2/46; A61F 2/4611; G16H 30/20; G16H 30/40; G16H 50/30; G16H 20/40; A61F 2/4455; G06T 7/20; G06T 7/0012; G06T 7/60; G06T 11/203; A61F 2002/4633; G16H 40/20

(57) ABSTRACT

Disclosed are methods, apparatuses and software products for graphic processing using a visual display system and image analysis for sizing of surgical implants in the planning and execution of spinal surgery, such as spinal fusion surgery of the cervical spine. The graphic processing includes determining a trajectory line for one or more target spine levels captured and measured by one or more measuring system to generate a 3D motion dataset for use in a range of diagnostic and therapeutic applications.

### CROSS-REFERENCE

This application claims the benefit of U.S. Provisional Application No. 63/022,639, filed May 11, 2020, which application is incorporated herein in its entirety by reference.

### BACKGROUND

As part of the diagnostic process for determining the cause of pain coming from a spinal joint, health care providers rely on an understanding of joint anatomy and joint mechanics when evaluating a subject's suspected joint problem and/or biomechanical performance issue. Currently available orthopedic diagnostic methods are capable of detecting a limited number of specific and treatable defects. These techniques include X-Ray, Mill, discography, and physical exams of the patient. In addition, spinal kinematic studies such as flexion/extension X-rays are used to specifically detect whether or not a joint has dysfunctional motion. These methods have become widely available and broadly adopted into the practice of treating joint problems and addressing joint performance issues.

What is needed are new devices, methods and software products for determining the target geometry for a level targeted for spinal surgery. Additionally, what is needed are devices, methods and software products for the safe operating range of spinal joints during surgery. Still other needs include devices, methods and software products for modeling and projecting various loads across spinal orthopedic implants.

Further, what is needed are methods, apparatuses and software products for graphic processing of spine images using a visual display system and for image analysis for sizing of surgical implants in the planning and execution of spinal surgery, such as spinal fusion surgery of the cervical spine.

### SUMMARY

Disclosed are methods, apparatuses and software products for processing in a visual display system which provides a tool for planning and execution of spine surgery. The methods and apparatuses allow for sizing of surgical implants during the planning and execution of the spine surgery.

Methods are disclosed in which computer graphic processing of image-derived measurements of intervertebral motion are used as an input. This computer graphic input dataset is derived from a fluoroscopic or X-ray image sequences of gross cervical bending of a patient as conducted during a diagnostic imaging session. This fluoroscopic imaging data (often referred to as a cine fluoroscopic sequence), or X-ray imaging data comprises a set of images taken during patient bending. The set of images is then processed to achieve a frame-to-frame registration of vertebral body positions across the sequence of individual frames comprising the cine fluoroscopic or X-ray image sequence. This frame-to-frame registrations comprises an x,y coordinate pair for each of the four corners associated with a four-point templating of a vertebral body on a lateral radiographic projection, for each vertebral body visible across the fluoroscopic image set.

An aspect of the disclosure is directed to image processing apparatuses. Suitable image processing apparatuses comprise one or more processors to select an input image of a target spine level having at least a first vertebral body and a second vertebral body; extract parameter information of at least one of the first vertebral body and the second vertebral body from the target spine level of the input image; derive a vertebral body motion from at least one of the first vertebral body and the second vertebral body of the input target spine level of the input image; determine a trajectory line for a motion of at least one of the first vertebral body and the second vertebral body of the target spine level; and analyze the vertebral body motion and trajectory line to determine a range of size parameters for surgical implant devices. Additionally, the one or more processors are configurable in some configurations to operate such that the one or more processors provide size parameter for surgical implant devices to at least one of a surgical planning system or an intra-operative system. The parameter information can be a box drawn from a four point markup of at least one of the first vertebral body and the second vertebral body. A first trajectory line can be provided which extends from a first corner point of a selected vertebral body of the least one of the first vertebral body and the second vertebral body. A second trajectory line can be provided which extends from a second corner point of the selected vertebral body. The parameter information can also be an outline of a first spinous process and a second spinous process of a vertebral body pair (e.g. cervical level or spinal level). Extension parameter information can be determined which corresponds to the first spinous process touching the second spinous process.

Another aspect of the disclosure is directed to methods of processing an image for use by an image processing apparatus having one or more processors comprising the steps of: selecting an input image of a target spine level having at least a first vertebral body and a second vertebral body; extracting parameter information of at least one of the first vertebral body and the second vertebral body from the target spine level of the input image; deriving a vertebral body motion from at least one of the first vertebral body and the second vertebral body of the input target spine level of the input image; determining a trajectory line for a motion of at least one of the first vertebral body and the second vertebral body of the target spine level; and analyzing the vertebral body motion and trajectory line to determine a range of size parameters for surgical implant devices. Additional steps can include, providing size parameter for surgical implant devices to at least one of a surgical planning system or an intra-operative system. More specifically, in some configurations, the parameter information can be a box drawn from a four point markup of at least one of the first vertebral body and the second vertebral body. A first trajectory line can be provided which extends from a first corner point of a selected vertebral body of the least one of the first vertebral body and the second vertebral body. Additionally, a second trajectory line can be provided that extends from a second corner point of the selected vertebral body. Parameter information can include, for example, an outline of a first spinous process and a second spinous process. Additionally, the method can include determining an extension parameter information which corresponds to the first spinous process touching the second spinous process.

Yet another aspect of the disclosure is directed to non-transitory computer readable medium having stored thereon a program for causing a computer to perform a method of processing an image comprising: selecting an input image of a target spine level having at least a first vertebral body and a second vertebral body; extracting parameter information of at least one of the first vertebral body and the second vertebral body from the target spine level of the input image; deriving a vertebral body motion from at least one of the first vertebral body and the second vertebral body of the input target spine level of the input image; determining a trajectory line for a motion of at least one of the first vertebral body and the second vertebral body of the target spine level; and analyzing the vertebral body motion and trajectory line to determine a range of size parameters for surgical implant devices. Additionally, the methods can comprise the step of providing size parameter for surgical implant devices to at least one of a surgical planning system or an intra-operative system. The parameter information can be a box drawn from a four point markup of at least one of the first vertebral body and the second vertebral body. A first trajectory line can be provided that extends from a first corner point of a selected vertebral body of the least one of the first vertebral body and the second vertebral body. A second trajectory line can be provided that extends from a second corner point of the selected vertebral body. In some configurations, the parameter information can include an outline of a first spinous process and a second spinous process. One or more processors can be provided for determining an extension parameter information which corresponds to the first spinous process touching the second spinous process.

Still another aspect of the disclosure is directed to a product comprising one or more tangible computer-readable non-transitory storage media comprising computer-executable instructions operable to, when executed by at least one processor, enable the at least one processor to cause the modeling and projecting system to select an input image of a target spine level having at least a first vertebral body and a second vertebral body; extract parameter information of at least one of the first vertebral body and the second vertebral body from the target spine level of the input image; derive a vertebral body motion from at least one of the first vertebral body and the second vertebral body of the input target spine level of the input image; determine a trajectory line for a motion of at least one of the first vertebral body and the second vertebral body of the target spine level; and analyze the vertebral body motion and trajectory line to determine a range of size parameters for surgical implant devices.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

U.S. Pat. No. 7,502,641 B2 issued Mar. 10, 2009 to Breen;

U.S. Pat. No. 8,676,293 B2 issued Mar. 18, 2014 to Breen et al.;

U.S. Pat. No. 8,777,878 B2 issued Jul. 15, 2014, to Deitz;

U.S. Pat. No. 9,138,163 B2 issued Sep. 22, 2015 to Deitz;

U.S. Pat. No. 9,277,879 B2 issued Mar. 8, 2016 to Deitz;

US 2016/0235479 A1 published Aug. 18, 2016 to Mosnier;

US 2016/0310374 A1 published Jul. 21, 2016 to Mosnier;

WO2015/040552 A1 published Mar. 26, 2015 to Mosnier et al; and

WO2015/056131 A1 published Apr. 23, 2015 to Mosnier et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C are block diagrams of a vertebral body pair in the cervical spine that illustrates how vertebral motion can be characterized as a “trajectory”;

FIGS. 2A-B illustrate a portion of the cervical spine (C2-C7) from a lateral view, with the spinous process of C3 and C4 having templates drawn during the marking up of radiographic images;

FIG. 3 is a simplified block diagram of a system used to produce three-dimensional motion measurements for spine levels; and

FIG. 4 is a simplified process diagram of a system used to produce three-dimensional motion measurements for spine levels.

### DETAILED DESCRIPTION

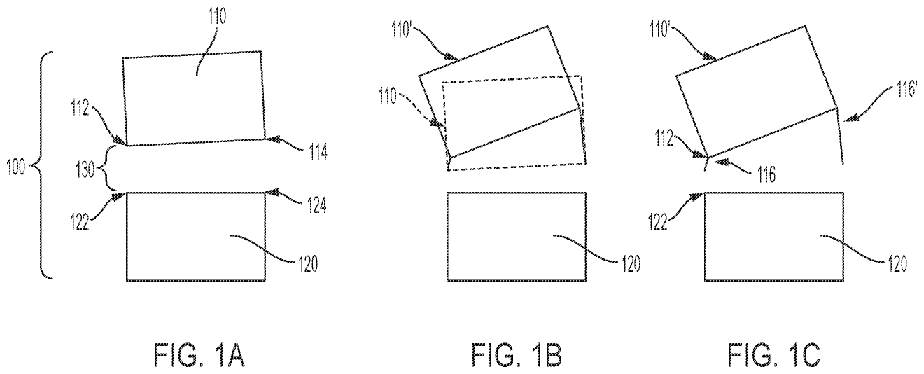

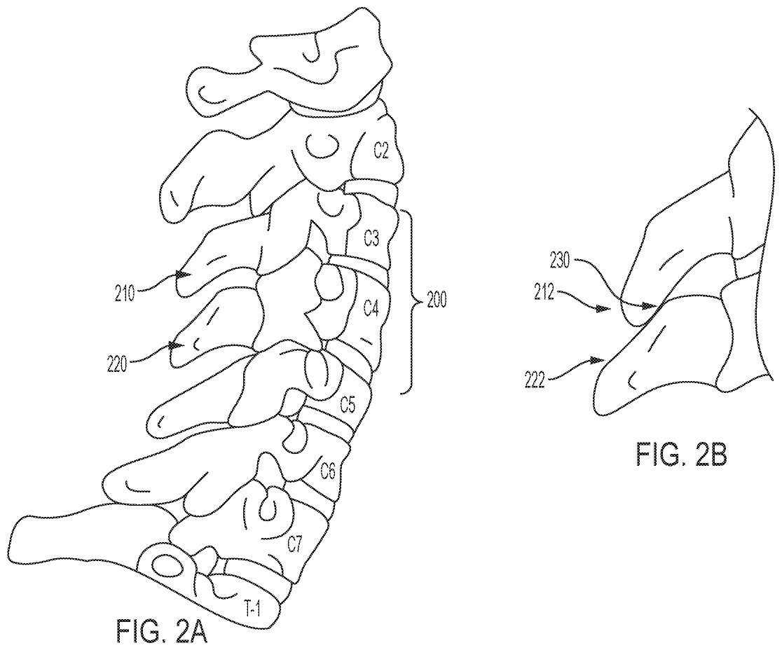

As depicted in FIGS. 1A-C, it is possible to determine a trajectory of the motion between a vertebral body pair 100 comprising two vertebral bodies, or a spine level, in a portion of the spine, for example, in the cervical portion of the spine at one or more spine levels. For ease of reference, the vertebral bodies are illustrated in FIGS. 1A-C as boxes. The first vertebral body 110, 110′, as illustrated, is a superior vertebral body in a vertebral body pair 100. The second vertebral body 120, as illustrated, is an inferior vertebral body. As will be appreciated by those skilled in the art, the vertebral bodies have a shape from a side view more closely captured in the illustration of FIG. 2A.

FIG. 1A depicts the vertebral body pair 100 in a first position with the first vertebral body 110 largely positioned in an aligned position over the second vertebral body 120. FIG. 1B and FIG. 1C depict this same vertebral body pair 100 shown in FIG. 1A including the first vertebral body 110 and the second vertebral body 120, where the first vertebral body 110 (shown in dashed lines) is in a second position (shown as first vertebral body 110′). The first vertebral body 110 has rotated from the first position shown in FIG. 1A into a second position shown in FIG. 1B and FIG. 1C.

The trajectory of motion of the first vertebral body 110 corresponds to changes in the disc height 130 separating two adjacent vertebral bodies in a spinal level, e.g., first vertebral body 110 and second vertebral body 120.

More specifically, the trajectory of motion can be determined by holding the two superior corner points 122, 124 of the inferior vertebral body (second vertebral body 120) of a vertebral body pair 100 at a spine level in a fixed position, and assessing the relative “trajectory” (shown as trajectory lines 116, 116′) of the two inferior corner points 112, 114 of the superior vertebral body (first vertebral body 110). relative to the two superior corner points of the inferior vertebral body from frame-to-frame across the cine fluoroscopic or X-ray imaging sequences.

As shown in FIG. 1B and FIG. 1C, the vertebral body pair 100 from FIG. 1A, including the first vertebral body 110′ (in the second position) and the second vertebral body 120, demonstrates that the first vertebral body 110′ has rotated from the first position shown in FIG. 1A into a second position shown in FIG. 1B and FIG. 1C. One or more trajectory lines 116, 116′, shown in FIG. 1C, illustrate the motion of the corner points between the two vertebral bodies. These trajectories represent the actual motion of the vertebral bodies across the image frames and can be described mathematically for a given vertebral body pair 100 or spine level, as well as statistically across spinal levels within a patient or across a plurality of patients at a given spinal level, e.g., C3-C4, C4-C5, C5-C6, etc.

A second measurement can be performed to measure the maximum size of an interbody device (not shown) for positioning within a disc space 130 between the first vertebral body 110 and the second vertebral body 120 based on a radiographic assessment of cervical intervertebral flexion/extension motion. Moreover, within the confines of the trajectory lines 116, 116′ described below, a “max extension” point can be determined. Determining the maximum extension point requires the user to template the edges of the spinous processes in the images (see FIG. 2A-B—a first spinous process 210 of C3 and a second spinous process 220 of C4 in a vertebral body pair 200 are the anatomical structures for a spine level that would be templated during image marked-up). As will be appreciated by those skilled in the art, this process can be repeated for additional spine levels in a patient as needed.

FIG. 2B depicts how these exemplar spinous processes 210, 220 would be marked-up. Each of the first spinous process 210 and the second spinous process 220 shown in FIG. 2A has a corresponding first spinous process outline 212 and a second spinous process outline 222. As apparent from FIG. 2B, the markup involves additional information beyond identifying four corner points of the vertebral body around the relatively square-shaped anterior vertebral body as illustrated in FIGS. 1A-C. The spinous process markup shown in FIGS. 2A-C allows the system to detect when, as a patient goes into extension, the lower edge of a first spinous process 210 touches an upper edge a second spinous process 220 at a touch point 230, which may be anywhere along the spinous process. When the edges of the adjacent spinous processes touch, e.g., at touch point 230, the location of touching is the point that represents an absolute maximum amount of lordosis and disc space 130 that a given vertebral body pair 100 should be assumed to be able to achieve the disc space 130 during patient movement without significant disruption of ligamentous or bony structures.

Once the templates for each spine level of interest are drawn, the maximum extension point and maximum interbody implant dimension can be determined in one of two ways: (1) for patients who bend completely such that in extension, the spinous processes touch or come very close to touching (i.e. the edges meet), then the maximum value is taken from the specific image at which the spinous processes are touching, and (2) for patients who do not bend completely, the trajectory is used in combination with the spinous processes edge markup data to project the maximum lordosis and/or disc height available at a level.

This method could be applied to determine the maximum dimensions possible (in terms of lordosis and/or disc height) for a cervical interbody device. Disc height can further be defined as anterior, midline, or posterior disc height. However in practice, many of the cervical levels that are targeted to receive fusions have collapsed and/or completely immobile disc. If this is the case, then it will not be possible to utilize the methodology above directly at a collapsed/immobile disc, however it will be possible to substitute that data for data drawn either from: (1) a normative assessment of neighboring levels within a patient, or (2) a normative assessment of the same level from other patients.

Once the implant sizing data is produced, the implant sizing data can be output for the user (via a device or paper report), transmitted or imported into a surgical planning system, or transmitted or imported into an intra-operative system.

Although described above with respect to C3-C4, these approaches could be applied to other spine levels in the cervical and/or the lumbar spine without departing from the scope of the disclosure. These approaches could also incorporate data drawn from MRI, X-ray, CT, and other imaging modalities to help make the trajectory setting process more accurate by providing information about many things including the facet orientation and locations and how the facet orientation changes during bending. These approaches could also factor in intervertebral translations (or intervertebral slip) that could alter the trajectory. When intervertebral translation is detected, the system could further seek to correct the motion trajectory and otherwise assist in projecting a corrected post-operative configuration that addresses anomalies related to intervertebral translation.

Additionally, one skilled in the art would appreciate that while fluoroscopic imaging allows for many frames of images data, effectively making it possible to determine the “trajectory” lines as described herein, in the case of plain-X-rays there may be only one or two data points. In this case, one skilled in the art would imagine many ways to interpolate a limited number of trajectory data points to produce a full projected trajectory dataset. Such ways could include using data from normative datasets of other patients as well as including data taken from other spine levels within a patient, or a combination of the two wherein a “best fit” trajectory line is determined via a statistical algorithm that considers a number of sources, both from within the patient and from other patients, which could be done on a patient specific basis considering such factors as age, gender, height, weight, co-morbidities, etc.

An additional aspect of the disclosure pertains to the underlying methods for producing intervertebral motion data. Intervertebral motion data is valuable clinically to spine practitioners in the assessment spinal pathologies, in particular spinal instability. Current X-ray technology is generally limited to making measurements of spinal motion in the sagittal or coronal plane. However, due to technical limitations, it is often impossible to assess axial motion of vertebral bodies from 2D medical images such as plain X-rays.

One skilled in the art would appreciate that skin surface marker-based methods are effective at measuring gross body motion, such as the rotation of joints or the movement of bodily structures such as the extremities or trunk. Systems such as OptiTrack® (manufactured by Natural Point, Inc., Corvallis, Oreg.) is an example of such measurement systems. Additionally, video and software registration based methods can be effective at measuring this gross body motion.

One object of the present disclosure is to provide methods and an apparatus for addressing the limitations associated with axial motion measurements from 2D plain X-rays. These methods and apparatus can incorporate a non-plain X-ray based motion capture measurement system—such as video capture systems with software registration or skin surface marker-based systems—for the purpose of capturing a gross anatomical motion in the axial plane, and combining this with plain X-ray based measurements of sagittal plane and coronal plane vertebral body motion. The purpose of this combination provides a process to correlate axial-plane data (from the motion capture systems) with coronal plane and sagittal plane data from plain X-rays to overcome the limitations of X-rays and produce anatomical motion data in all three anatomical planes (sagittal plane, coronal plane, and transverse plane).

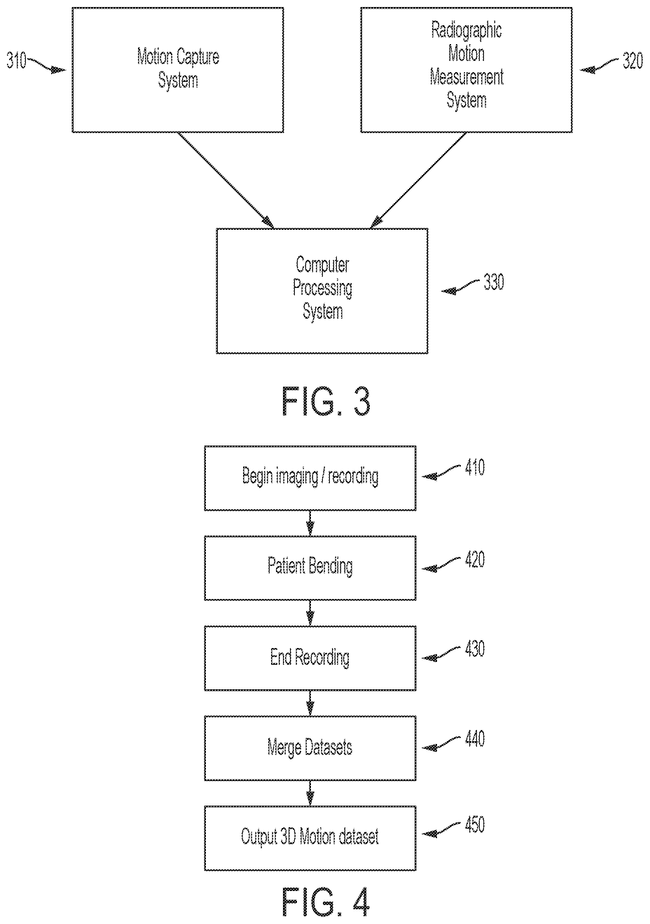

The apparatus shown in FIG. 3 depicts a system that incorporates: (1) an apparatus associated with a motion capture system 310, (2) an apparatus associated with a radiographic motion measurement system 320, and (3) a computer processing system 330 configured to aggregate the data from one or more motion capture systems 310 and one or more radiographic motion measurement systems 320, and perform calculations required to produce an output comprised of diagnostic data. The method involved includes: (1) using the motion capture system 310 to measure gross motion during patient spinal bending in the sagittal plane and/or coronal plane (this gross motion would occur during imaging, and the resulting images are processed to derive inter-vertebral motion data); (2) using the motion capture system 310 to measure the gross motion during patient axial bending; (3) optionally capturing radiographic images via the radiographic motion measurement system 320 at the starting and/or ending points of patient axial bending, then process these images to produce relative assessments of intervertebral axial rotation; and (4) using the computer processing system 330 to correlate the data from the motion capture system 310 and the radiographic motion measurement system 320 to produce one or more assessments of spinal bending.

This process is described more formally in FIG. 4 which shows how the integral system produces three-dimensional intervertebral motion output. The process starts by getting a patient positioned relative to two apparatuses and ready to begin bending. The first apparatus is a motion capture system 310. The second apparatus is the radiographic motion measurement system 320. When the patient is ready to begin bending, imaging and data recording is initiated 410 on the motion capture system 310, and the radiographic motion measurement system 320 (when used). As the patient bends 420, the motion capture system 310 and, optionally, the radiographic motion measurement system 320 record the motion of the patient and create an associated dataset for the recording. After the patient has completed one or more bends, the data recording ends 430 (i.e., stop recording) on the motion capture system 310 and the radiographic motion measurement system 320 (when used). The captured data is provided to the computer processing system 330 where the captured data is merged into a single dataset 440 during a processing step. During the processing step, the gross motion from the motion capture system 310 may need to be interpolated at the inter-vertebral level. Once the data from the motion capture systems is merged, and there is a complete three-dimensional dataset for each level imaged, this data is then output 450 as a 3D motion dataset to another system for use in a range of diagnostic and therapeutic applications. One skilled in the art will recognize that for the radiographic motion measurement system 320, there may need to be two patient bending datasets recorded and merged. For example, there may need to be a separate bend for flexion and extension vs. left/right bending. The step at which all data is merged into a single dataset 440 could therefore incorporate data from multiple bending planes.

The systems and methods according to aspects of the disclosed subject matter may utilize a variety of computer and computing systems, communications devices, networks and/or digital/logic devices for operation. Each may, in turn, be configurable to operate so that the systems utilize a suitable computing device that can be manufactured with, loaded with and/or fetch from some storage device, and then execute, instructions that cause the computing device to perform a method according to aspects of the disclosed subject matter.

In engaging the systems and methods according to aspects of the disclosed subject matter, a user may engage in one or more use sessions. A use session may include a training session for the user.

The systems and methods according to aspects of the disclosed subject matter may utilize a variety of computer and computing systems, communications devices, networks and/or digital/logic devices for operation. Each may, in turn, be configurable to operate so that the systems utilize a suitable computing device that can be manufactured with, loaded with and/or fetch from some storage device, and then execute, instructions that cause the computing device to perform a method according to aspects of the disclosed subject matter.

A computing device can include without limitation a mobile user device such as a mobile phone, a smart phone and a cellular phone, a personal digital assistant (“PDA”), such as an iPhone®, a tablet, a laptop and the like. In at least some configurations, a user can execute a browser application over a network, such as the internet, to view and interact with digital content, such as screen displays. A display includes, for example, an interface that allows a visual presentation of data from a computing device. Access could be over or partially over other forms of computing and/or communications networks. A user may access a web browser, e.g., to provide access to applications and data and other content located on a website or a webpage of a website.

A suitable computing device may include a processor to perform logic and other computing operations, e.g., a stand-alone computer processing unit (“CPU”), or hard wired logic as in a microcontroller, or a combination of both, and may execute instructions according to its operating system and the instructions to perform the steps of the method, or elements of the process. The user's computing device may be part of a network of computing devices and the methods of the disclosed subject matter may be performed by different computing devices associated with the network, perhaps in different physical locations, cooperating or otherwise interacting to perform a disclosed method. For example, a user's portable computing device may run an app alone or in conjunction with a remote computing device, such as a server on the Internet. For purposes of the present application, the term “computing device” includes any and all of the above discussed logic circuitry, communications devices and digital processing capabilities or combinations of these.

Certain embodiments of the disclosed subject matter may be described for illustrative purposes as steps of a method that may be executed on a computing device executing software, and illustrated, by way of example only, as a block diagram of a process flow. Such may also be considered as a software flow chart. Such block diagrams and like operational illustrations of a method performed or the operation of a computing device and any combination of blocks in a block diagram, can illustrate, as examples, software program code/instructions that can be provided to the computing device or at least abbreviated statements of the functionalities and operations performed by the computing device in executing the instructions. Some possible alternate implementation may involve the function, functionalities and operations noted in the blocks of a block diagram occurring out of the order noted in the block diagram, including occurring simultaneously or nearly so, or in another order or not occurring at all. Aspects of the disclosed subject matter may be implemented in parallel or seriatim in hardware, firmware, software or any combination(s) of these, co-located or remotely located, at least in part, from each other, e.g., in arrays or networks of computing devices, over interconnected networks, including the Internet, and the like.

The instructions may be stored on a suitable “machine readable medium” within a computing device or in communication with or otherwise accessible to the computing device. As used in the present application a machine readable medium is a tangible storage device and the instructions are stored in a non-transitory way. At the same time, during operation, the instructions may at times be transitory, e.g., in transit from a remote storage device to a computing device over a communication link. However, when the machine readable medium is tangible and non-transitory, the instructions will be stored, for at least some period of time, in a memory storage device, such as a random access memory (RAM), read only memory (ROM), a magnetic or optical disc storage device, or the like, arrays and/or combinations of which may form a local cache memory, e.g., residing on a processor integrated circuit, a local main memory, e.g., housed within an enclosure for a processor of a computing device, a local electronic or disc hard drive, a remote storage location connected to a local server or a remote server access over a network, or the like. When so stored, the software will constitute a “machine readable medium,” that is both tangible and stores the instructions in a non-transitory form. At a minimum, therefore, the machine readable medium storing instructions for execution on an associated computing device will be “tangible” and “non-transitory” at the time of execution of instructions by a processor of a computing device and when the instructions are being stored for subsequent access by a computing device.

As will be appreciated by those skilled in the art, the systems and methods disclosed are configurable to operate so that the systems send a variety of messages when alerts are generated. Messages include, for example, SMS and email.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An image processing apparatus comprising one or more processors configured to
select an input image of a target spine level having at least a first vertebral body and a second vertebral body;
extract parameter information of at least one of the first vertebral body and the second vertebral body from the target spine level of the input image;
derive a vertebral body motion from at least one of the first vertebral body and the second vertebral body of the input image of the target spine level;
determine a trajectory line for a motion of at least one of the first vertebral body and the second vertebral body of the target spine level; and
analyze the vertebral body motion and the trajectory line to determine a range of size parameters for surgical implant devices.

2. The image processing apparatus of claim 1 comprising one or more processors to provide size parameter for surgical implant devices to at least one of a surgical planning system and an intra-operative system.

3. The image processing apparatus of claim 1 comprising one or more processors wherein the parameter information is a box drawn from a four point markup of at least one of the first vertebral body and the second vertebral body.

4. The image processing apparatus of claim 1 comprising one or more processors wherein a first trajectory line extends from a first corner point of a selected vertebral body of the at least one of the first vertebral body and the second vertebral body.

5. The image processing apparatus of claim 4 comprising one or more processors wherein a second trajectory line extends from a second corner point of the selected vertebral body.

6. The image processing apparatus of claim 1 comprising one or more processors wherein the parameter information is an outline of a first spinous process and a second spinous process.

7. The image processing apparatus of claim 6 comprising one or more processors to determine an extension parameter information which corresponds to the first spinous process touching the second spinous process.

8. A method of processing an image for use by an image processing apparatus having one or more processors, the method comprising:
selecting an input image of a target spine level having at least a first vertebral body and a second vertebral body;
extracting parameter information of at least one of the first vertebral body and the second vertebral body from the target spine level of the input image;
deriving a vertebral body motion from at least one of the first vertebral body and the second vertebral body of the input target spine level of the input image;
determining a trajectory line for a motion of at least one of the first vertebral body and the second vertebral body of the target spine level; and
analyzing the vertebral body motion and trajectory line to determine a range of size parameters for surgical implant devices.

9. The method of processing of claim 8 comprising providing size parameter for surgical implant devices to at least one of a surgical planning system an an intra-operative system.

10. The method of processing of claim 8 wherein the parameter information is a box drawn from a four point markup of at least one of the first vertebral body and the second vertebral body.

11. The method of processing of claim 8 wherein a first trajectory line extends from a first corner point of a selected vertebral body of the least one of the first vertebral body and the second vertebral body.

12. The method of processing of claim 11 wherein a second trajectory line extends from a second corner point of the selected vertebral body.

13. The method of processing of claim 8 wherein the parameter information is an outline of a first spinous process and a second spinous process.

14. The method of processing of claim 13 comprising one or more processors for determining an extension parameter information which corresponds to the first spinous process touching the second spinous process.

15. A non-transitory computer readable medium having stored thereon a software program for causing a computer to perform a method of processing an image, the method comprising:
selecting an input image of a target spine level having at least a first vertebral body and a second vertebral body;
extracting parameter information of at least one of the first vertebral body and the second vertebral body from the target spine level of the input image;
deriving a vertebral body motion from at least one of the first vertebral body and the second vertebral body of the input target spine level of the input image;
determining a trajectory line for a motion of at least one of the first vertebral body and the second vertebral body of the target spine level; and
analyzing the vertebral body motion and trajectory line to determine a range of size parameters for surgical implant devices.

16. The non-transitory computer readable medium of claim 15 comprising providing size parameter for surgical implant devices to at least one of a surgical planning system and an intra-operative system.

17. The non-transitory computer readable medium of claim 15 wherein the parameter information is a box drawn from a four point markup of at least one of the first vertebral body and the second vertebral body.

18. The non-transitory computer readable medium of claim 15 wherein a first trajectory line extends from a first corner point of a selected vertebral body of the least one of the first vertebral body and the second vertebral body.

19. The non-transitory computer readable medium of claim 18 wherein a second trajectory line extends from a second corner point of the selected vertebral body.

20. The non-transitory computer readable medium of claim 15 wherein the parameter information is an outline of a first spinous process and a second spinous process.

21. The non-transitory computer readable medium of claim 20 comprising one or more processors for determining an extension parameter information which corresponds to the first spinous process touching the second spinous process.
